(19) United States
(12) Patent Application Publication
(10) Pub. No.: US 2026/0053496 A1
(43) Pub. Date: Feb. 26, 2026

(54) Auxiliary Device For Passing Suture Through Tissue And Method

(71) Applicant: Arthrex, Inc., Naples, FL (US)

(72) Inventor: Peter J. DREYFUSS, Naples, FL (US)

(73) Assignee: Arthrex, Inc., Naples, FL (US)

(21) Appl. No.: 19/270,762

(22) Filed: Jul. 16, 2025

Related U.S. Application Data
(60) Provisional application No. 63/686,913, filed on Aug. 26, 2024.

(51) Int. Cl. A61B 17/04

(52) U.S. Cl. CPC A61B 17/0482; A61B 2017/0445

(57) ABSTRACT

Surgical constructs, assemblies, kits and methods of tissue fixation are disclosed. A surgical guide includes a cannula with a distal section having a proximal slot, a distal slot, and a notch opening at the distal end. The surgical guide is configured to securely hold tissue while a penetrating device (such as a suture passer) slidingly and removably passes through the surgical guide to penetrate tissue and retrieve suture.

### CROSS-REFERENCE TO RELATED APPLICATIONS

This application claims priority to US Provisional Application No. 63/686,913 filed on Aug. 26, 2025, the disclosure of which is hereby incorporated by reference in its entirety herein.

### BACKGROUND

The present disclosure relates to the field of surgery and, more particularly, to surgical instruments and methods of surgical repairs.

### SUMMARY

Surgical instruments, assemblies, and methods are disclosed. An auxiliary surgical instrument is in the form of a cannula with a distal section having a proximal slot, a distal slot, and a notch opening at the distal end. The instrument is configured to securely hold tissue while a penetrating device (such as a suture passer) slidingly passes through the instrument to penetrate tissue and retrieve suture.

Methods of surgeries are also disclosed. In an embodiment, a method of arthroscopic surgery is conducted with a medical device configured to hold soft tissue while a penetrating device (such as a suture passer) passes through a cannulation of the medical device to grab and/or retrieve suture. The method employs a single portal for tissue grasping and suture retrieval.

### BRIEF DESCRIPTION OF THE DRAWINGS

FIG. 1 illustrates a perspective distal view of a surgical instrument.

FIG. 2 illustrates a right-side view of the surgical instrument of FIG. 1.

FIGS. 3-10 illustrate steps of an exemplary method of tissue repair with a surgical instrument.

### DETAILED DESCRIPTION

The present disclosure provides surgical instruments, assemblies, kits, and methods of surgery.

A surgical instrument can provide suture passing capabilities through a soft tissue that is securely engaged and held by the surgical instrument. A surgical instrument can be a suture passing guide that allows soft tissue to be securely positioned and held by the guide while a penetrating instrument can slide through the guide to grab and/or retrieve suture from the soft tissue.

Methods of endoscopic or arthroscopic surgeries are also disclosed. An exemplary method includes inter alia the steps of: (i) employing a surgical guide to position and hold soft tissue within a slot of the surgical guide; and (ii) retrieving a flexible coupler through the soft tissue with a penetrating instrument that is slidingly passed through a cannulation of the surgical guide. The method can further include conducting steps (i) and (ii) simultaneously. The method can further include conducting steps (i) and (ii) using a single portal approach. A penetrating instrument can be a suture passer. A penetrating instrument can be any arthroscopic and open suture passer or penetrator that passes a flexible strand such as FiberWire® suture through tissue(s).

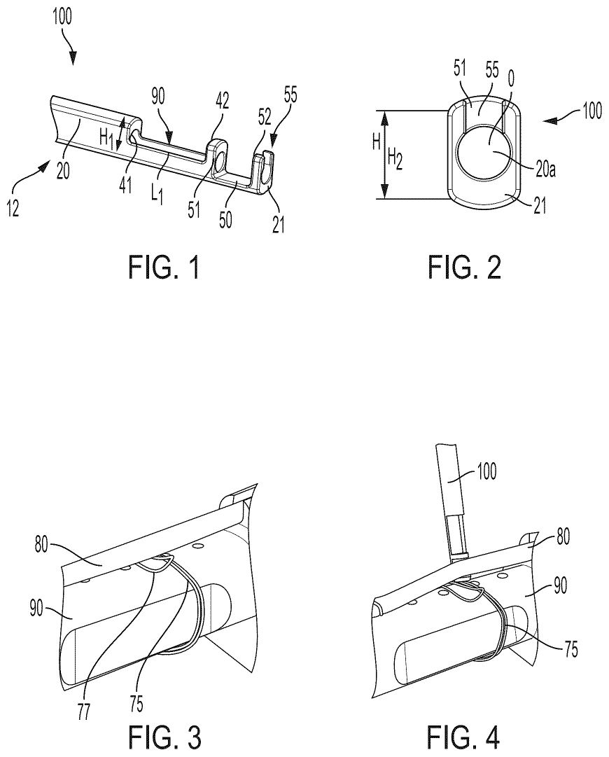

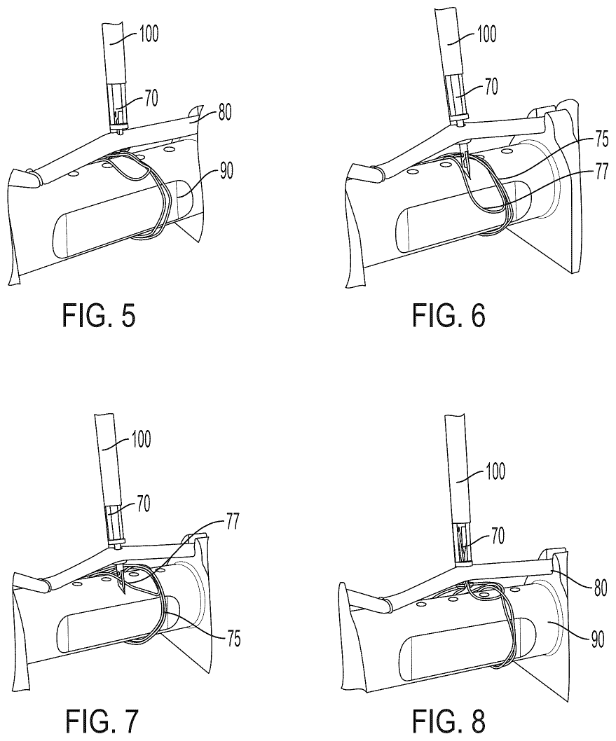

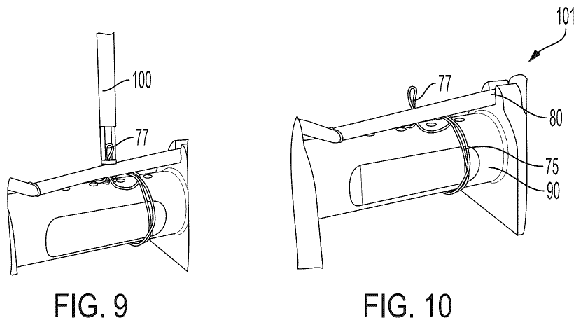

Referring now to the drawings, where like elements are designated by like reference numerals, FIGS. 1 and 2 illustrate exemplary surgical guide 100 (auxiliary device 100; surgical instrument 100; device 100). FIGS. 3-10 illustrate steps of an exemplary method of reconstructive surgery with surgical guide 100 of the present disclosure.

Surgical guide 100 described below can be employed, for example, as part of a surgical procedure directed at repairing soft tissue injuries (e.g., rotator cuff injuries). To that end, surgical guide 100 is configured to grab, hold, and secure soft tissue and to allow another instrument to pass through the surgical guide, to penetrate the soft tissue secured by the surgical guide and to control (grab/retrieve/pass) one or more flexible strands (such as suture) through the soft tissue. Surgical guide 100 can be used alone, or in combination with additional surgical instruments such as drills, penetrators and/or suture passers/retrieval instruments as part of various surgical guide assemblies.

FIGS. 1 and 2 illustrate perspective and side views of a distal end of exemplary surgical guide 100. Surgical guide 100 can be part of surgical assembly 101 (shown in FIGS. 5-8) which includes surgical guide 100 secured to soft tissue 80 and a penetrating device 70 slidable within a cannula of surgical guide 100.

Surgical guide 100 of FIG. 1 comprises a handle (not shown) at the proximal end of the guide and a shaft, tube, or cannula 20 at distal end 12 of the guide 100. Cannula 20 has a hollow, elongated body with a proximal end (attached to the handle) and an open distal end 22. The cannula body has a hollow through bore 20a that extends along a longitudinal axis 10 of the guide 100. The cannula body can be rigid or partially rigid and can be formed of various materials such as metals, alloys, polymers, plastics, or combinations thereof. The tube or cannula 20 can have various cross-sections such as circular or elliptical, among many others. The cannula body can be constructed with enough strength and rigidity to withstand being forcibly advanced in the vicinity of soft tissue and/or bone during a surgical procedure.

Distal end 12 of guide 100 includes a proximal slot 40 (cut-out 40) provided adjacent a distal slot 50 (cut-out slot 50). Proximal slot 40 is an elongated opening 40 (groove 40; depression 40; aperture 40) formed within cannula 20 and extending along the longitudinal axis 10 of the surgical guide 100. Proximal slot 40 has a length L1, a height H1, and an overall geometry similar to that of a semicircle. Proximal slot 40 extends between proximal wall 41 and distal wall 42. Through bore 20a of cannula 20 extends through the proximal slot 40 and through the proximal and distal walls 41, 42. The dimensions and geometry of proximal slot 40 can vary depending on the jaw configuration of the penetrator/suture passer to be used with the surgical guide 100. The proximal slot 40 allows the jaw of the penetrator/suture passer to open (or fully open) to release suture within the proximal slot, as detailed below.

Distal slot 50 is positioned adjacent the proximal slot 40 and extending along the longitudinal axis 10 of the guide 100. Distal slot 50 is also an elongated opening 50 (50; groove 50; depression 50; aperture 50) formed within cannula 20. Distal slot 50 has a length L2, a height H2, and an about rectangular cross-sectional view. Distal slot 50 extends between proximal wall 51 and a distal wall 52. Through bore 20a of cannula 20 extends through the distal slot 50 and through the walls 51, 52. In an embodiment, length L2 of distal slot 50 is smaller than length L1 of proximal slot 40. In an embodiment, height H2 of the distal slot 50 is greater than height H1 of proximal slot 40. In an embodiment, height H2 of the distal slot 50 is about equal to height H of cannula 20. The dimensions and geometry of distal slot 50 can vary depending on the tissue to be grasped and held during the surgical procedure. The distal slot 50 functions to prevent the tissue 80 from trying to push away.

Most distal end 21 of guide 100 is provided with a top notch or notch opening 55 (aperture 55; slot 55; gap 55; slit 55; cut 55), to allow passage of a flexible coupler such as suture, and as described below. Notch opening 55 can be dimensioned to allow easy passage and sliding of one or more flexible couplers therethrough. As shown in FIG. 1, most distal end 21 also communicates with the through bore 20a to allow full passage of another surgical instrument, and as detailed below.

Reference is now made to FIGS. 3-10 which illustrate exemplary steps of a tissue repair 101 (FIG. 10) with surgical guide 100 and exemplary instrument 70. The sequence of steps detailed below is for an exemplary method of holding tissue 80 while passing through the tissue with a penetrating instrument 70 (such as a suture passer, for example) using a single portal:

FIG. 3 illustrates soft tissue 80 (first tissue 80; tendon 80; labrum 80; graft 80) adjacent another tissue 90 (second tissue 90; hard tissue 90; bone 90). Exemplary suture loop 77 of flexible strand 75 is shown adjacent tissue 90 and as part of a tissue repair, for example, a cerclage repair.

FIG. 4: Surgical guide 100 is positioned around soft tissue 80 so that the soft tissue 80 is secured in the distal slot 50 of the surgical guide 100.

FIG. 5: A penetrating instrument 70 (e.g., an Arthrex Nano BirdBeak® penetrator/suture passer 70 or a similar suture passer/penetrator device) is brought down through the cannula 20 of surgical guide 100 forming assembly 110. Instrument 70 can be provided with a sharp tip to penetrate the tissue 80 which is positioned within the distal slot 50. FIG. 5 shows the tip of instrument 70 within the proximal slot 40 and without yet penetrating soft tissue 80 secured within distal slot 50.

FIG. 6: Instrument 70 is illustrated passed through soft tissue 80 and passed through walls 41, 42, 51, 52 and extending along, and concentrically with, longitudinal axis 10 of surgical guide 100.

FIG. 7: Once the penetrating instrument 70 is through the tissue 80, the penetrating instrument 70 can grab a suture and retrieve it (pull it back) through the tissue 80. FIG. 7 illustrates instrument 70 capturing loop 77 of flexible strand 75 secured around tissue 90. Loop 77 can be a continuous, adjustable, flexible, self-locking, knotless loop. Loop 77 can have a fixed or adjustable perimeter and length. Loop 77 can be a suture loop as part of biceps tenodesis. Flexible strand 75 can be suture.

FIG. 8: Captured loop 77 is pulled back through soft tissue 80 with instrument 70.

FIG. 9: Captured loop 77 is released in the proximal slot 40 of the guide 100.

FIG. 10: Instrument 70 and surgical guide 100 are removed from the surgical site. During removal of the guide 100, suture loop 77 retained within the proximal slot 40 of the guide 100 is pulled back (slides back) through opening O between walls 42, 51 (opening O is shown in FIG. 2) while surgical guide 100 is pulled out of tissue 80. In this manner, suture loop 77 remains positioned on soft tissue 80, as part of repair 101 and for use in any additional surgical steps. The notch opening 55 allows the suture 75, 77 to travel through the notch freeing the suture 75, 77 as surgical guide 100 is removed from the soft tissue 80 positioned in its distal slot 50.

The surgical guide 100 allows the surgeon to hold tissue 80 while the tissue is penetrated with another device (such as instrument 70) using a single portal. Upon passing the suture 75, 77 can be released on either side of the pass. The surgical guide 100 prevents suture tangling during a surgical procedure, aids in suture management, and speeds up the overall technique.

Surgical guide 100 detailed above is a medical instrument with applicability to various open or arthroscopic procedures including procedures for re-approximating bone to bone or soft tissue to bone, for example, shoulder rotator cuff repairs, capsulolabral reconstruction, SLAP repairs, as well as ankle, knee, elbow or foot repairs, among many others.

Surgical guide 100 detailed above can be part of a surgical kit, assembly, or system to simplify the surgeon's task of selecting a specific instrument and to aid in the overall surgical procedure. As detailed above, surgical guide 100 can be part of assembly 110 that includes a penetrator and/or suture passer 70. A surgical kit for an arthroscopic surgical repair can include one or more surgical guides 100; assemblies 110; any suture passers and/or penetrators and/or similar devices, as well as drills or bone-penetrating devices such as various cutting devices. The surgical kit can also include fixation devices (such as anchors, screws, suture-button constructs, plates, etc.) and flexible members (such as flexible couplers 75 and flexible loops 77) to be employed in conjunction with the tissue repairs conducted with surgical guide 100. Surgical guide 100 can be reusable or disposable. Surgical guide 100 can be formed by injection molding or by any other methods known in the art.

Fixation devices can be anchors, for example, knotted anchors, knotless anchors, or all-suture anchors, or any devices that confer secure attachment and fixation of soft tissue over bone. A fixation device can be a knotless anchor such as a two-piece Arthrex PushLock® anchor, disclosed in US 7,329,272, or an Arthrex SwiveLock® anchor, disclosed in US 8,012,174 and US 9,005,246, the disclosures of both of which are fully incorporated by reference in their entirety herein. A fixation device can be an all-suture soft anchor (soft suture anchor) provided with a soft anchor sleeve (sheath, tubular member) with two open ends and one or more flexible shuttling strands extending through the soft anchor sleeve (sheath). Flexible strands can extend through the sleeve in similar or different directions and/or orientations and/or locations. The flexible sleeve with the one or more shuttling strands is secured into or onto bone, and the strands allow passing of additional flexible strands such as sutures or tapes to pass over soft tissue and be secured into bone to approximate soft tissue to bone. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

A surgical guide 100 comprises: a tube or cannula 20 having a proximal end, a distal end 12, and a longitudinal axis 10; and a handle attached to the proximal end of the tube or cannula 20, wherein the tube or cannula 20 further includes a proximal slot 40, a distal slot 50, and a notch 55 provided at a most distal end 21 of the tube or cannula 20.

The distal slot retains soft tissue and prevents it from trying to push away. A surgical guide 100 can be an integrated device that can allow an additional instrument 70 to slide through the guide and to penetrate the soft tissue 80 captured and retained within the distal slot 50.

A surgical guide assembly 110 comprises: a surgical guide 100; and a penetrating instrument 70 slidably and removably attached to the surgical guide 100. The penetrating instrument 70 advances through (slides through) a through bore 20a of cannula 20 of the surgical guide 100 along a longitudinal axis 10 of the guide 10 and about parallel to the longitudinal axis 10 of the guide. In an embodiment, a longitudinal axis of the penetrating instrument 70 is about parallel and concentric (coincides) with longitudinal axis 10 of the surgical guide 100.

Methods of endoscopic or arthroscopic surgeries are also disclosed.

An exemplary method includes inter alia the steps of: (i) employing a surgical guide 100 to position and hold soft tissue 80 within a slot 50 of the surgical guide 100; and (ii) retrieving at least one flexible coupler 75, 77 through the soft tissue 80 with a penetrating instrument 70 that is slidingly and removably passed through a cannulation 20 of the surgical guide 100. The method can further include conducting steps (i) and (ii) simultaneously. The method can further include conducting steps (i) and (ii) using a single portal. A penetrating instrument 70 can be a suture passer, a penetrator, a suture retriever, or combinations thereof. A penetrating instrument 70 can be any arthroscopic and open suture passer, suture passing instrument, or penetrator that passes at least one flexible strand 75, 77 such as FiberWire® suture through tissue 80. The at least one flexible strand 75, 77 can include a plurality of flexible strands 75 and/or loops 77 that can be passed at a same surgical site and location, or at different locations.

Another exemplary method includes inter alia the steps of: (i) capturing soft tissue 80 within a distal slot 50 of a surgical guide 100; (ii) inserting a penetrator 70 through a cannula 20 of the surgical guide 100; (iii) passing the penetrator 70 through the soft tissue 80; (iv) capturing a flexible strand or loop 77, 75 with the penetrator 70; (v) retrieving the flexible strand or loop 77, 75 through the soft tissue 80; and (vi) removing the penetrator 70 and the surgical guide 100. The method can further include the steps of attaching the flexible strand to another tissue 90; and securing the flexible strand or loop 77, 75 to at least one fixation device. The fixation device can be a knotted or knotless anchor. The fixation device can be a hard body anchor, a soft body anchor, or an all-suture soft anchor. The flexible strand or loop 77, 75 can be a flexible coupler such as suture or suture loop, or combinations thereof.

The flexible strands or loops 77, 75 can be formed of any flexible material such as suture or tape, or combination of suture and tape. The suture can be in the form of any known suture construct, such as multifilament, braided, knitted, woven suture, or including fibers of ultrahigh molecular weight polyethylene (UHMWPE) or the FiberWire® suture (disclosed in US 6,716,234, the disclosure of which is hereby incorporated by reference in its entirety herein). The tape can be formed of suture tape, for example, Arthrex FiberTape®, which is a high strength suture tape that is braided and rectangular-like in cross section and as disclosed in US 7,892,256, the disclosure of which is hereby incorporated by reference in its entirety herein. Flexible strands or loops 77, 75 can include any soft, flexible strand of material. Flexible strands or loops 77, 75 can consist essentially of elastic suture.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed is:

1. A surgical guide comprising:
a cannula with a through bore, a longitudinal axis, a proximal end, and a distal end;
a proximal slot and a distal slot both located at the distal end of the cannula and both communicating with the through bore, wherein the proximal slot has a first configuration, and wherein the distal slot has a second configuration; and
a notch opening provided at a most distal end of the cannula, wherein the notch opening communicates with the distal slot and with the through bore.

2. The surgical guide of claim 1, wherein the distal slot is configured to capture and retain soft tissue to be sutured.

3. The surgical guide of claim 2, wherein the cannula is configured to allow a penetrating instrument to slidingly pass through the cannula and penetrate the soft tissue to be sutured.

4. The surgical guide of claim 3, wherein the penetrating instrument is a suture passer.

5. The surgical guide of claim 4, wherein the suture passer captures suture to be passed through the soft tissue and pulls the suture through the soft tissue.

6. The surgical guide of claim 5, wherein a longitudinal axis of the suture passer coincides with the longitudinal axis of the cannula.

7. The surgical guide of claim 5, wherein the notch opening is configured to allow the suture to be freed as the surgical guide is removed from the soft tissue.

8. The surgical guide of claim 1, wherein the surgical guide is used in arthroscopic or endoscopic surgery.

9. The surgical guide of claim 1, wherein the surgical guide is used in biceps reconstruction.

10. The surgical guide of claim 1, wherein the first configuration is about semicircular and the second configuration is about rectangular.

11. A surgical kit, comprising:
a guide comprising a cylindrical cannulated shaft with a through bore, a longitudinal axis, a proximal end and a distal end; handle located at the proximal end; a proximal slot and a distal slot both located at the distal end of the cylindrical cannulated shaft and both communicating with the through bore, wherein the proximal slot has a first configuration, and wherein the distal slot has a second configuration different from the first configuration; and a notch opening provided at a most distal end of the cylindrical cannulated shaft, wherein the notch opening communicates with the distal slot and with the through bore; and
a suture passer.

12. The surgical kit of claim 11, wherein the suture passer slidingly and removably passes through the cylindrical cannulated shaft, and wherein a longitudinal axis of the suture passer coincides with the longitudinal axis of the guide.

13. The surgical kit of claim 11, further comprising a fixation device.

14. The surgical kit of claim 13, wherein the fixation device is an anchor, a button, a screw, or a plate.

15. The surgical kit of claim 13, wherein the fixation device is a soft suture anchor comprising a flexible tubular sleeve or sheath and a plurality of flexible strands extending through a passage of the flexible tubular sleeve or sheath.

16. A method of passing suture through soft tissue, comprising:
securing the soft tissue with a surgical instrument comprising a cylindrical cannulated shaft with a through bore, a longitudinal axis, a proximal end and a distal end; a proximal slot and a distal slot both located at the distal end of the cylindrical cannulated shaft and both communicating with the through bore, wherein the proximal slot has a first configuration, and wherein the distal slot has a second configuration; and a notch opening provided at a most distal end of the cylindrical cannulated shaft, wherein the notch opening communicates with the distal slot and with the through bore; and
passing a suture passer through the cylindrical cannulated shaft and through the soft tissue.

17. The method of claim 16, further comprising the steps of:
positioning the surgical instrument around the soft tissue so that the soft tissue is secured within the distal slot of the cylindrical cannulated shaft;
inserting the suture passer through the cylindrical cannulated shaft and through the soft tissue;
capturing at least one flexible strand with the suture passer; and
pulling the at least one flexible through the soft tissue.

18. The method of claim 17, further comprising releasing the at least one flexible strand in the proximal slot of the surgical instrument.

19. The method of claim 17, further comprising freeing the at least one flexible strand in the notch opening of the surgical instrument while the surgical instrument is removed from the soft tissue secured within the distal slot.

20. The method of claim 17, wherein the at least one flexible strand is suture or suture tape.

21. The method of claim 17, wherein the at least one flexible strand includes a loop.

22. The method of claim 16, wherein the soft tissue is tendon, ligament, or graft.

23. The method of claim 16 employing a single surgical portal.

24. The method of claim 16, wherein the soft tissue is part of a rotator cuff repair.
